(19) United States
(12) Patent Application Publication
(10) Pub. No.: US 2026/0191684 A1
(43) Pub. Date: Jul. 9, 2026

(54) OPHTHALMIC SURGICAL HANDPIECE AND OPHTHALMIC SURGICAL SYSTEM COMPRISING SUCH A HANDPIECE

(71) Applicant: Carl Zeiss Meditec Cataract Technology, Inc., Reno, NV (US)

(72) Inventors: Luke W. Clauson, Reno, NV (US); Viktor Schaitan, Reno, NV (US); Scott Chamness, Reno, NV (US)

(21) Appl. No.: 19/551,442

(22) Filed: Feb. 26, 2026

Related U.S. Application Data
(63) Continuation of application No. PCT/US24/44682, filed on Aug. 30, 2024.

(30) Foreign Application Priority Data
Sep. 1, 2023 (DE) 10 2023 123 606.7

(51) Int. Cl. A61F 9/007; A61M 1/00; A61M 39/00; A61M 39/22

(52) U.S. Cl. CPC A61F 9/00745; A61M 1/774; A61M 39/22; A61M 2039/0009; A61M 2202/0007; A61M 2202/0014; A61M 2202/04; A61M 2205/0216; A61M 2205/103; A61M 2205/3331; A61M 2205/50; A61M 2210/0612

(57) ABSTRACT

An ophthalmic surgical handpiece includes a hollow needle, an irrigation fluid pump having a first port as a fluid inlet and a second port as a fluid outlet, an aspiration fluid pump configured to deliver aspiration fluid, the aspiration fluid pump being connected to a fluid line which is arranged to receive aspiration fluid conveyed with the hollow needle, and to a fluid line which is arranged to discharge the aspiration fluid at its distal end into a collection container, and a drive device including a drive shaft and a gear, wherein the drive shaft is configured to drive the irrigation fluid pump and the aspiration fluid pump together with a drive apparatus which is configured to be coupled to the drive device, and wherein the drive shaft is configured to drive the hollow needle in a predetermined direction with the gear.

### CROSS REFERENCE TO RELATED APPLICATIONS

This application is a continuation application of international patent application PCT/US24/44682, filed Aug. 30, 2024, designating the United States and claiming priority to German application 10 2023 123 606.7, filed Sep. 1, 2023, and the entire content of both applications is incorporated herein by reference.

### TECHNICAL FIELD

The disclosure relates to an ophthalmic surgical handpiece and an ophthalmic surgical system comprising such a handpiece.

### BACKGROUND

There are several surgical techniques for treating an opacity of the lens of the eye, which is referred to in medicine as a cataract. The most common technique is phacoemulsification, which uses an ophthalmic surgical handpiece that has a thin hollow needle that is inserted into the lens of the eye and caused to vibrate. The vibrating hollow needle cuts and emulsifies the lens in its immediate vicinity in such a way that the resulting lens particles can be aspirated by a pump starting from the distal end of the hollow needle and passing through the hollow needle. A rinsing fluid (irrigation fluid) is supplied, and the lens particles and fluid are aspirated through an aspiration fluid line to a collection container. Once the lens has been completely emulsified and removed, a new artificial lens can be inserted into the empty capsular bag, allowing a patient treated in this way to regain good vision.

During aspiration, it can happen that a lens particle is so large that it cannot flow through the hollow needle and clogs the distal end of the hollow needle. To cause the particle to break, the vacuum can be increased using the pump. If the particle then breaks, the blockage of the hollow needle ends, which is called occlusion break. Occlusion break causes a relatively sharp change in pressure to the range of the usual suction pressure in a relatively short time. This pressure fluctuation can cause the intraocular pressure to also fluctuate significantly and eventually cause the eye to collapse. This can lead to severe eye damage, which should be avoided.

### SUMMARY

It is an object of the disclosure to provide an ophthalmic surgical handpiece and an ophthalmic surgical system including such a handpiece, with which the removal of an eye lens can be performed safely and reliably.

The object is achieved by a ophthalmic surgical handpiece and an ophthalmic surgical system as described herein.

The ophthalmic surgical handpiece includes a hollow needle designed as a cutting instrument, an irrigation fluid pump which is configured to deliver irrigation fluid, the irrigation fluid pump having a first port as a fluid inlet to which a first fluid line is connected, the first fluid line being configured to be fluidically connected to an irrigation fluid container, and having a second port as a fluid outlet to which a second fluid line is connected, the second fluid line having a distal end arranged to allow the irrigation fluid to flow out into a distal region of the hollow needle, an aspiration fluid pump, which is configured to deliver aspiration fluid, wherein the aspiration fluid pump is connected to a third fluid line, which is configured to receive aspiration fluid conveyed with the hollow needle, and is connected to a fourth fluid line, which is configured to discharge the aspiration fluid at its distal end into a collection container, and a drive device including a drive shaft and a gear wherein the drive shaft is configured to drive the irrigation fluid pump and the aspiration fluid pump together with a drive apparatus which is configured to be coupled to the drive device and wherein the drive shaft is configured to drive the hollow needle in a predetermined direction with the gear.

In accordance with an aspect of the disclosure, the ophthalmic surgical handpiece includes an irrigation fluid pump. Irrigation fluid may flow from an irrigation fluid container by way of the first fluid line to a first port of the irrigation fluid pump, said first port forming a fluid inlet for the irrigation fluid pump. The irrigation fluid pump pumps the irrigation fluid to a second port of the irrigation fluid pump, which forms a fluid outlet. From there, the irrigation fluid can flow out into the distal region of the hollow needle via the second fluid line. Since the irrigation fluid pump is not—as in conventional machines for phacoemulsification-located in a console at a distance of at least two meters tube length away from the ophthalmic surgical handpiece, but directly in the ophthalmic surgical handpiece, there is a relatively short distance between the second port of the irrigation fluid pump and the distal end of the second fluid line connected to it. This means that the required irrigation fluid can be supplied very quickly after an occlusion break is detected, so that the risk of an eye collapsing is significantly reduced.

The disclosure is based on the idea that it is unavoidable if, after an occlusion break, a strong suction in the eye causes the intraocular pressure to drop sharply. However, to ensure that such negative pressure in the eye does not cause the eye to collapse, according to the disclosure an irrigation fluid pump is provided in the handpiece and thus very close to the eye. Thus, there is no attempt to prevent the negative pressure present in the aspiration fluid line from reaching the anterior chamber of the eye after an occlusion break, but rather the negative consequences of this negative pressure are prevented by the rapid provision of the irrigation fluid.

If not only the irrigation fluid pump but also the aspiration fluid pump is located in the handpiece, this means that the third fluid line has a relatively short length. When the aspiration fluid pump is activated, this means that a rapid change in suction pressure can be achieved. It is thus possible that both the supply of the irrigation fluid and the aspiration of the aspiration fluid can be controlled in a relatively short time, thus achieving a high level of safety and reliability of a lens extraction.

According to an aspect of the disclosure, the drive device includes a drive shaft and a gear which is configured to drive the irrigation fluid pump and the aspiration fluid pump together with a drive apparatus which is configured to be coupled to the drive device. This makes it possible to drive and control both pumps with only one drive apparatus, so that the supply of irrigation fluid and the discharge of aspiration fluid can be synchronized. This increases safety and reliability when removing the eye lens.

The irrigation fluid pump and the aspiration fluid pump are two separate pumps that can be driven together but controlled differently.

The rotary movement of the drive shaft can also be used to drive the hollow needle with the gear. This enables a very compact design for the handpiece. The drive shaft also makes it possible for the aspiration fluid pump and the irrigation fluid pump to be arranged one behind the other along a longitudinal axis of the drive shaft, so that the handpiece has a small diameter. This makes the handpiece easy for a user to grip and ergonomic.

According to a further aspect of the disclosure, an overrunning clutch is provided on the drive shaft between the aspiration fluid pump and the irrigation fluid pump. This makes it possible to drive the aspiration fluid pump in the reverse direction while at the same time not driving the irrigation fluid pump. Such a reversal in the running direction of the aspiration fluid pump is useful because, in the event of occlusion at the distal end of the hollow needle by a particle, this particle can be removed from the distal end of the hollow needle by flowing out the aspiration fluid. This can be helpful in cases of severe occlusion. In addition, such a reversal in the running direction of the aspiration fluid pump is advantageous if tissue of the iris or capsular bag is inadvertently aspirated during aspiration and the tissue is to be removed again by the hollow needle. In this case, a stop of the irrigation fluid pump means that no further irrigation fluid is supplied to the anterior chamber of the eye, so that the intraocular pressure is not increased further.

It is further possible that the irrigation fluid pump is configured to deliver a first volumetric flow of irrigation fluid and the aspiration fluid pump is configured to deliver a second volumetric flow of aspiration fluid, wherein the amount of the first volumetric flow is higher than the amount of the second volumetric flow. Such an embodiment is advantageous to compensate for an unavoidable loss of fluid at the puncture site of the hollow needle on the eye, or leakage, so that the risk of pumping the eye empty can be kept low. If the first volume flow is larger than the second volume flow, this can also be advantageous in the case of occlusion break. In this case, a relatively large amount of fluid is drawn out of the anterior chamber of the eye in a relatively short period of time, so the eye could collapse. If more irrigation fluid is then supplied by the higher second volume flow, this reduces the risk of the eye collapsing.

In particular, the amount of the first volume flow is in the range of 1.01 to 1.3 of the amount of the second volume flow.

Typically, the second fluid line is made of a material with a hardness larger than 55 ShA. The second fluid line is thus not as soft as a normally used silicone hose and can expand only very little or not at all in the event of a strong pressure fluctuation in its circumference. This increases the certainty that no pressure fluctuation is induced or amplified by the fluid line thus designed. Particularly typically, the second fluid line is formed from a hard plastic (thermoplastic or duroplastic) or from a material comprising metal.

According to a further aspect of the disclosure, the second fluid line has a length of less than 20 cm, and typically less than 3 cm. This allows irrigation fluid to be delivered relatively quickly to the distal region of the hollow needle, so that a pressure fluctuation in the eye can be compensated relatively quickly.

It is advantageous if an irrigation fluid reservoir is provided in the second fluid line, which is configured to receive and store irrigation fluid. If, due to an occlusion break at the hollow needle in the anterior chamber of the eye, a strong pressure drop or even a negative pressure should occur, so that a rapid pressure equalization should take place, the irrigation fluid reservoir can provide an irrigation fluid contained therein, which is drawn into the anterior chamber due to the negative pressure in the anterior chamber of the eye without any control and without any assistance by a pump. Typically, the irrigation fluid reservoir is a rigid container having therein an elastic membrane body which is in a relaxed state with no irrigation fluid in the irrigation fluid reservoir and is in an elastically compressed state when the irrigation fluid reservoir is filled with irrigation fluid and is configured to elastically relax upon a decrease in fluid pressure in the irrigation fluid reservoir and thereby actively transport irrigation fluid out of the irrigation fluid reservoir.

It is further possible that a fifth fluid line is provided connecting the first fluid line and the second fluid line to one another, and wherein a first pressure relief valve is provided in the fifth fluid line. If, during an occlusion of the hollow needle, the irrigation fluid pump is not in operation and an occlusion suddenly occurs, irrigation fluid can flow from the irrigation fluid container through the first fluid line and from there through the fifth fluid line and thereafter through the second fluid line. This can be done, for example, at a pressure of 30 mmHg, which is determined by the pressure relief valve. This ensures a constant, but not too high, volume flow of irrigation fluid. The pressure relief valve prevents too much irrigation fluid from flowing out of the irrigation fluid container and prevents the container from emptying too quickly and preventing too much irrigation fluid from being supplied to the eye. The fifth fluid line with the first pressure relief valve bypasses the irrigation fluid pump. This can further increase safety when removing an eye lens.

Typically, the irrigation fluid pump and/or aspiration fluid pump is a vane pump, which particularly typically has a rotor that has vanes made of a rubber-elastic material. Such a vane pump with rubber-elastic vanes is particularly reliable compared to a vane pump with rigid vanes guided in radial slots. It is possible for lens particles contained in the aspiration fluid to enter such slots and impede or block vane movement. This can lead to undesirable fluctuations in the volume flow. Rubber-elastic vanes, on the other hand, do not move in any slot, so that no clogging or even blocking of a rotor is to be expected and thus a safe and reliable delivery of a volume flow is possible.

The ophthalmic surgical system according to an aspect of the disclosure includes the previously described handpiece, a drive apparatus configured to be coupled to the drive device, a control unit configured to control the drive apparatus, an irrigation fluid container with irrigation fluid, and a collection container configured to collect aspiration fluid.

### BRIEF DESCRIPTION OF THE DRAWINGS

The invention will now be described with reference to the drawings wherein:

FIG. 1 shows a schematic representation of a first exemplary embodiment of an ophthalmic surgical handpiece coupled to an irrigation fluid container, a collection container and a drive apparatus; and

FIG. 2 shows a schematic representation of a second exemplary embodiment of an ophthalmic surgical handpiece coupled to an irrigation fluid container, a collection container and a drive apparatus.

### DESCRIPTION OF EXEMPLARY EMBODIMENTS

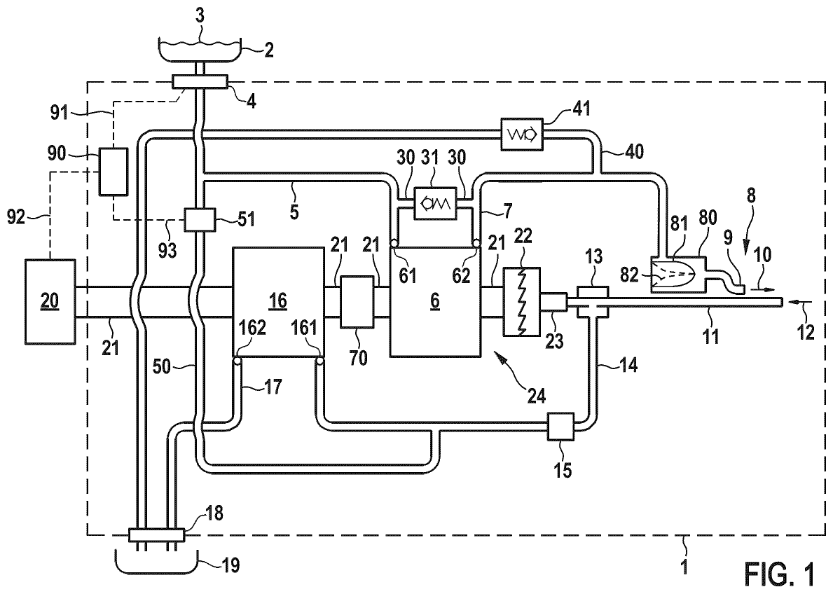

FIG. 1 shows a schematic representation of a first exemplary embodiment of an ophthalmic surgical handpiece 1. An irrigation fluid container 2 contains an irrigation fluid 3, which can be supplied to a first fluid line 5 in the handpiece 1 with a first connecting piece 4 of the handpiece 1. The first connecting piece 4 may additionally include a valve, which may release or interrupt the inflow of irrigation fluid 3. The valve may be controlled by a control unit 90 arranged in the handpiece 1 with a first control mechanism or a first control line 91. The control mechanism or first control line 91 may be mechanical or electrical. The distal end of the first fluid line 5 is connected to a first port 61 of an irrigation fluid pump 6, so that the irrigation fluid 3 enters the irrigation fluid pump 6.

The irrigation fluid pump 6 can supply the irrigation fluid 3 to a second port 62 of the irrigation fluid pump 6 so that it enters a second fluid line 7 and can flow to a distal end 9 and from the distal end 9, see arrow 10. The distal end 9 is arranged to be located in a distal region 8 of a hollow needle 11 of the handpiece 1.

The hollow needle 11 is configured to be pierced through a cornea of an eye and into the anterior chamber of the eye. Further, it is configured to aspirate fluid at its distal end, see arrow 12. The fluid is irrigation fluid and crushed lens particles, which together is referred to as aspiration fluid. The suction pressure in the hollow needle 11 is generated by an aspiration fluid pump 16, which is connected at a third port 161 to a third fluid line 14, which in turn is fluidly connected to the hollow needle 11. The third fluid line 14, together with the line in the hollow needle 11, can be referred to as the aspiration fluid line.

A fluid chamber 13 can be provided in the area of the proximal end of the hollow needle 11, in which crushed lens particles can collect. The fluid chamber 13 can have a filter for this purpose, for example. This is advantageous, as it ensures that no particles in the third fluid line 14 can lead to a blockage and thus a safe suction of the fluid can be achieved.

Should an excessive pressure fluctuation occur in the third fluid line 14, for example after an occlusion break, the aspiration of aspiration fluid can be reduced or completely interrupted with a valve 15 in the third fluid line 14. The valve 15 can be controlled by the control unit 90. However, it is also possible for the valve 15 to limit or interrupt the volume flow through the fluid line 14 independently and without actuation depending on the amount of a pressure fluctuation.

The aspiration fluid pump 16 is provided with a fourth port 162, which is connected to a fourth fluid line 17, in order to supply the aspiration fluid along this fluid line 17 to a second connecting piece 18 on the ophthalmic surgical handpiece 1. From there, the aspiration fluid can be collected in a collection container 19.

In this exemplary embodiment, the irrigation fluid pump 6 and the aspiration fluid pump 16 are driven by a drive apparatus 20, which is, for example, an energy storage device or a drive motor. The drive apparatus 20 is typically arranged outside the handpiece 1 and can be coupled to a drive device 24 arranged in the handpiece 1. The drive device 24 includes a drive shaft 21 and a gear 22. The drive apparatus 20 is arranged to be controlled by the control unit 90 with a second control mechanism or a second control line 92. The control mechanism or second control line 92 may be mechanical or electrical. When the drive apparatus 20 is controlled by the control unit 90, the drive apparatus 20 may drive the drive shaft 21 which causes, for example, a rotating motion in the aspiration fluid pump 16 and the irrigation fluid pump 6. The drive shaft 21 is provided with the gear 22 at the output of the irrigation fluid pump 6, wherein the gear produces a transformation of the rotational motion of the drive shaft 21 into a translational motion. The translational motion may act directly on the hollow needle 11, wherein the gear 22 is arranged to typically achieve a translational forward motion and backward motion of the hollow needle 11. The gear 22 may be a pilgrim step gear or a saw-tooth rotary disc cam and cam follower. The gear 22 may also be arranged to provide a torsional movement of the hollow needle 11. When the hollow needle 11 encounters an ocular lens with a forward and backward motion or with a torsional motion, the ocular lens may thereby be cut and crushed into individual particles.

Instead of acting directly on the hollow needle 11, the gear 22 may also act indirectly on the hollow needle with a coupling piece 23. This can be useful to achieve an ideal transmission of the translatory motion to the hollow needle 11.

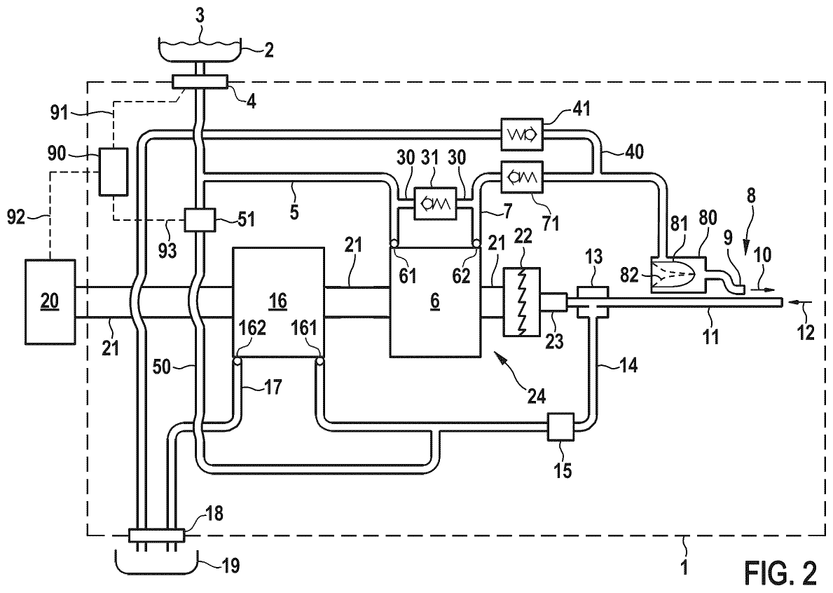

If the hollow needle 11 becomes clogged at its distal end by a particle that is not crushed either by a strong negative pressure in the hollow needle 11 or by an oscillating movement of the hollow needle 11, the aspiration fluid pump 16 can be driven in the opposite direction by the drive apparatus 20. Then the aspiration fluid pump 16 does not have a suction effect, but a delivery effect. This results in that an overpressure is generated in the third fluid line 14, which forces out a particle at the distal end of the hollow needle 11. During this process, it is not practical to continue to feed irrigation fluid through the irrigation fluid pump 6 into the eye anterior chamber, so that the irrigation fluid pump 6 is uncoupled from the drive shaft 21 rotating in the reverse direction with an overrunning clutch 70. The torque is thus transmitted in one direction of rotation only. As an alternative to the overrunning clutch 70 in a second exemplary embodiment of the handpiece 1, a third pressure relief valve 71 can be provided in the second fluid line 7, which interrupts an intake of irrigation fluid from the distal region 8 into the irrigation fluid pump 6, see FIG. 2. When the direction of rotation of the drive apparatus 20 is reversed, the irrigation fluid pump 6 acts as a suction pump which, however, cannot suck out the irrigation fluid located downstream of the third pressure relief valve 71, which is located between the third pressure relief valve 71 and the distal area 8 in the second fluid line 7, due to the third pressure relief valve 71.

A fifth fluid line 30 may be provided between the first fluid line 5 and the second fluid line 7, in which a first pressure relief valve 31 is provided. For example, if the irrigation fluid pump 6 is not delivering irrigation fluid because of an occlusion at the hollow needle 11 and suddenly the occlusion breaks, not enough irrigation fluid may be delivered to the anterior chamber of the eye until the irrigation fluid pump is started. To ensure that some irrigation fluid is always available and flowing in the second fluid line 7, irrigation fluid 3 can be permanently supplied into the second fluid line 7 with the fifth fluid line 30. The pressure relief valve 31 ensures that even if the hydrostatic pressure in the first fluid line 5 is high, the irrigation fluid 3 is only introduced into the second fluid line 7 at a predetermined pressure, which is lower than the hydrostatic pressure specified by the position of the irrigation fluid container 2. The pressure relief valve 31 can be designed to limit a fluid pressure of maximum 100 mmHg at its inlet to a fluid pressure of maximum 40 mmHg, typically 30 mm Hg, at its outlet.

If the fluid pressure in the second fluid line 7 is nevertheless too high, the irrigating fluid 3 can be discharged in the direction of the collection container 19 through a bypass line 40, which is provided with a second pressure relief valve 41.

If the negative pressure in the third fluid line 14 is too high and should be reduced somewhat, for example when polishing a capsule bag, irrigation fluid 3 can be supplied to the third fluid line 14 with a sixth fluid line 50. This lowers the negative pressure, and this can be controlled by a valve 51. The valve 51 can be controlled by the control unit 90 with a third control mechanism or a third control line 93. If a strong pressure decrease causes a relatively large amount of fluid to be sucked out of the eye during occlusion at the distal end of the hollow needle 11, an irrigation fluid reservoir 80 containing a membrane body 81 can be provided in the second fluid line 7. When the irrigation fluid reservoir 80 is filled with irrigation fluid 3, the membrane body 81 is compressed with the irrigation fluid 3, see dashed line with reference sign 82.

When a strong pressure decrease is created in the anterior chamber of the eye, the membrane body 81 in the compressed state can form back into its original shape (see reference sign 81), thereby exerting an overpressure on the irrigation fluid 3 in the irrigation fluid reservoir 80. This allows the irrigation fluid 3 to enter the eye even more quickly, further reducing the risk of collapse of the eye.

The ophthalmic surgical handpiece 1 with the hollow needle 11 can be used to emulsify a lens. The hollow needle can also be partially surrounded by a sleeve and used as a vitrectomy probe.

LIST OF REFERENCE NUMERALS

| | |
|---|---|
| 1 | handpiece |
| 2 | irrigation fluid container |
| 3 | irrigation fluid |
| 4 | first connecting piece |
| 5 | first fluid line |
| 6 | irrigation fluid pump |
| 7 | second fluid line |
| 8 | distal region of hollow needle |
| 9 | distal end of second fluid line |
| 10 | flow of irrigation fluid |
| 11 | hollow needle |
| 12 | suction direction for aspiration fluid |
| 13 | fluid chamber |
| 14 | third fluid line |
| 15 | valve |
| 16 | aspiration fluid pump |
| 17 | fourth fluid line |
| 18 | second connecting piece |
| 19 | collection container |
| 20 | drive apparatus |
| 21 | drive shaft |
| 22 | gear |
| 23 | coupling piece |
| 24 | drive device |
| 30 | fifth fluid line |
| 31 | first pressure relief valve |
| 40 | bypass line |
| 41 | second pressure relief valve |
| 50 | sixth fluid line |
| 51 | valve |
| 61 | first port of the irrigation fluid pump |
| 62 | second port of the irrigation fluid pump |
| 70 | overrunning clutch |
| 71 | third pressure relief valve |
| 80 | irrigation fluid reservoir |
| 81 | membrane body in relaxed state |
| 82 | membrane body in compressed state |
| 90 | control unit |
| 91 | first control line |
| 92 | second control line |
| 93 | third control line |
| 161 | third port |
| 162 | fourth port |

What is claimed is:

1. An ophthalmic surgical handpiece, comprising:
a hollow needle configured as a cutting instrument;
an irrigation fluid pump configured to deliver irrigation fluid, the irrigation fluid pump having a first port as a fluid inlet to which a first fluid line is connected, the first fluid line being configured to be fluidically connected to an irrigation fluid container and having a second port as a fluid outlet to which a second fluid line is connected, the second fluid line having a distal end arranged to allow the irrigation fluid to flow out into a distal region of the hollow needle;
an aspiration fluid pump configured to deliver aspiration fluid, wherein the aspiration fluid pump is connected to a third fluid line which is configured to receive aspiration fluid conveyed with the hollow needle, and which is connected to a fourth fluid line which is configured to discharge the aspiration fluid at its distal end into a collection container; and
a drive device having a drive shaft and a gear, wherein the drive shaft is configured to drive the irrigation fluid pump and the aspiration fluid pump together with a drive apparatus configured to be coupled to the drive device, and
wherein the drive shaft is configured to drive the hollow needle in a predetermined direction with the gear.

2. The ophthalmic surgical handpiece according to claim 1, further comprising:
an overrunning clutch arranged on the drive shaft between the aspiration fluid pump and the irrigation fluid pump.

3. The ophthalmic surgical handpiece according to claim 1, wherein the irrigation fluid pump is configured to deliver a first volumetric flow of irrigation fluid,
wherein the aspiration fluid pump is configured to deliver a second volumetric flow of aspiration fluid, and
wherein the amount of the first volumetric flow is higher than the amount of the second volumetric flow.

4. The ophthalmic surgical handpiece according to claim 1, wherein the second fluid line is made of a material having a hardness larger than 55 ShA.

5. The ophthalmic surgical handpiece according to claim 1, wherein the second fluid line has a length of less than 20 cm.

6. The ophthalmic surgical handpiece according to claim 1, further comprising:
an irrigation fluid reservoir arranged in the second fluid line and configured to receive irrigation fluid,
wherein the irrigation fluid reservoir has an elastic membrane body which is in a relaxed state with no irrigation fluid in the irrigation fluid reservoir and which is in an elastically compressed state when the irrigation fluid reservoir is filled with irrigation fluid, and
wherein the elastic membrane body is configured to elastically relax upon a decrease in fluid pressure in the irrigation fluid reservoir and thereby to actively transport irrigation fluid out of the irrigation fluid reservoir.

7. The ophthalmic surgical handpiece according to claim 1, further comprising:
a fifth fluid line which connects the first fluid line with the second fluid line, and
a first pressure relief valve arranged in the fifth fluid line.

8. The ophthalmic surgical handpiece according to claim 1, wherein the irrigation fluid pump and/or aspiration fluid pump is a vane pump.

9. An ophthalmic surgical system, comprising:
a handpiece according to claim 1;
a drive apparatus configured to be coupled to the drive device;
a control unit configured to control the drive apparatus;
an irrigation fluid container with irrigation fluid; and
a collection container configured to collect aspiration fluid.
